(19) United States
(12) Patent Application Publication
(10) Pub. No.: US 2026/0027337 A1
(43) Pub. Date: Jan. 29, 2026

(54) TRANSDERMAL DRUG DELIVERY DEVICES AND METHODS

(71) Applicant: Morningside Venture Investments Limited, Monaco (MC)

(72) Inventors: Andrew L. JOHNSTON, Redwood City, CA (US); Michael P. SCHALLER, Louisville, CO (US); Bryce PETERSON, Redwood City, CA (US); Kevin W. GELSTON, Moraga, CA (US)

(21) Appl. No.: 19/347,556

(22) Filed: Oct. 1, 2025

Related U.S. Application Data
(63) Continuation of application No. 18/662,905, filed on May 13, 2024.
Application No. 18/662,905 is a continuation of application No. 17/703,910, filed on Mar. 24, 2022, now Pat. No. 12,042,614.
Application No. 17/703,910 is a continuation of application No. 16/473,981, now Pat. No. 11,285,306.
(60) Provisional application No. 62/443,421, filed on Jan. 6, 2017.

(51) Int. Cl. A61M 35/00; A61K 9/70; A61M 5/142; A61M 5/145

(52) U.S. Cl. CPC A61M 35/00; A61K 9/703; A61M 5/14248; A61M 5/145; A61M 5/1454; A61M 2005/14268; A61M 2005/14533; A61M 2205/103; A61M 2205/106; A61M 2205/502; A61M 2205/8206

(57) ABSTRACT

A transdermal drug delivery device includes a reservoir, a transdermal membrane, a piston, a control rod, a spring, and a rotational cam. The reservoir is configured to hold a formulation. The transdermal membrane is configured to allow the formulation from the reservoir to pass therethrough. The piston is configured to move into the reservoir. The control rod is attached to the piston and includes a plurality of teeth thereon. The spring is configured to apply force to the control rod in the direction of the reservoir. The rotational cam has a first camming surface and a second camming surface that are configured to engage with the plurality of teeth. The rotational cam, when rotated, is configured to disengage the first camming surface from a first tooth of the plurality of teeth, thereby allowing the spring to advance the piston into the reservoir to expel the formulation onto the transdermal membrane.

### CROSS REFERENCE TO RELATED APPLICATIONS

This patent application is a continuation of U.S. patent application Ser. No. 18/662,905, titled “TRANSDERMAL DRUG DELIVERY DEVICES AND METHODS,” filed on May 13, 2024, now U.S. Patent Application Publication No. 2024/0408362, which is a continuation of U.S. patent application Ser. No. 17/703,910, titled “TRANSDERMAL DRUG DELIVERY DEVICES AND METHODS,” filed on Mar. 24, 2022, now U.S. Pat. No. 12,042,614, which is a continuation of U.S. patent application Ser. No. 16/473,981, titled “TRANSDERMAL DRUG DELIVERY DEVICES AND METHODS,” filed on Jun. 26, 2019, now U.S. Pat. No. 11,285,306, which is the U.S. National Stage Entry of International Patent Application No. PCT/US2018/012568, titled “TRANSDERMAL DRUG DELIVERY DEVICES AND METHODS,” filed on Jan. 5, 2018, which claims priority to U.S. Provisional Application No. 62/443,421, filed Jan. 6, 2017, and titled “TRANSDERMAL DRUG DELIVERY DEVICES AND METHODS,” which applications are hereby incorporated herein by reference in their entireties.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present application relates generally to devices and methods for providing a formulation to a patient transdermally.

### BACKGROUND

Medicinal drugs are given to people to manage or improve their health for a variety of reasons, such as to manage nicotine or another addiction or dependency, to manage pain, or to prevent or treat a medical condition or disease such as diabetes, Parkinson's disease, or ulcerative colitis.

Some medicinal drugs are rapidly metabolized by the body. Multiple doses of the drug over a period of time are therefore often needed to provide a desired effect. In addition to having the desired preventative or therapeutic effects, medicinal drugs can also have negative side-effects on the body that can range from irritating to life-threatening. A person's body can also develop tolerance to a drug and experience a diminished response to the drug after taking it for a period of time and require higher doses to have an effect, resulting in increased drug use and additional side-effects. It is therefore beneficial to a person taking a drug to dose the drug properly to reduce tolerance and/or side-effects.

Transdermal drug delivery is one way to deliver medicinal drugs to a patient. However, current transdermal drug delivery systems can be improved by any one of: a size reduction, a smaller volume, a lower profile to reduce the height and/or cross-sectional foot print, a reduced weight, reduction of moving parts, a reduction of expensive parts, a decreased cost, a reduced engagement force between the reusable part and drug cartridge, and more accurate dosage delivery.

Accordingly, a transdermal drug delivery system that provides some or all of these improvements is desired.

### SUMMARY OF THE DISCLOSURE

The present invention relates generally to systems for delivering a formulation transdermally and methods for using the systems to deliver the formulation.

In general, in one embodiment, a transdermal drug delivery device includes a reservoir, a transdermal membrane, a piston, a control rod, a spring, and a rotational cam. The reservoir is configured to hold a formulation therein. The transdermal membrane is configured to allow the formulation from the reservoir to pass therethrough. The piston is configured to move into the reservoir. The control rod is attached to the piston and includes a plurality of teeth thereon. The spring is configured to apply force to the control rod in the direction of the reservoir. The rotational cam has a first camming surface and a second camming surface that are configured to engage with the plurality of teeth. The rotational cam, when rotated, is configured to disengage the first camming surface from a first tooth of the plurality of teeth, thereby allowing the spring to advance the piston into the reservoir to expel the formulation onto the transdermal membrane.

This and other embodiments can include one or more of the following features. The transdermal drug delivery device can further include a motor configured to rotate the rotational cam. The device can include a first part that includes the reservoir, membrane, piston, control rod, spring, and rotational cam and a second part that includes the motor and a power source. The first and second parts can be configured to engage and disengage from one another. The first part can be disposable, and the second part can be reusable. The first part can further include a storage latch that is configured to hold the spring away from the control rod during storage. The storage latch can be configured to release the spring when the first and second parts are engaged. The transdermal drug delivery device can further include a user interface and a display. The control rod and plunger can have a substantially linear configuration. The reservoir can have a substantially linear shape. The control rod and plunger can have a curved configuration. The reservoir can have a semi-annular shape. A spacing between the plurality of teeth on the control rod can define a teeth spacing pattern, and the teeth spacing pattern can correspond to a drug delivery profile of the transdermal drug delivery device. The teeth spacing pattern can have a substantially uniform spacing. A distance between the first and second camming surfaces can be substantially equivalent to a distance between adjacent teeth. The teeth spacing pattern can have a non-uniform spacing. The drug delivery profile can correspond to a circadian rhythm or a bio-synchronous pattern of a patient using the transdermal drug delivery device. The first and second camming surfaces can be circumferentially offset from one another. The first and second camming surfaces may not overlap circumferentially. The device can be configured such that, after the first camming surface disengages from the first tooth and pushes the spring to push the control rod and piston into the reservoir, the second camming surface engages with a second tooth of the plurality of teeth to stop the piston from moving further into the reservoir. The cam can be rotatable in a first direction to cause the first camming surface to disengage with the first tooth and the second camming surface to engage with the second tooth. The cam can be rotatable in a second direction to cause the second camming surface to disengage from the second tooth and cause the piston to move further into the reservoir to expel more of the formulation. The cam can be rotatable alternately in the first and second directions to allow sequential bolus deliveries of the formulation. The cam can be rotatable a first distance in a first direction to cause the first camming surface to disengage with the first tooth and the second camming surface to engage with the second tooth. The cam can be rotatable a second distance in the first direction to cause the second camming surface to disengage from the second tooth and cause the piston to move further into the reservoir to expel more of the formulation. The spring can be configured to apply a force of about 12 N or less to move the control rod. The transdermal drug delivery can further include a pathway between the reservoir and the transdermal membrane. The transdermal drug delivery device can further include a valve along the pathway between the reservoir and the transdermal membrane. The formulation can be selected from the group consisting of: nicotine, Acamprosate, Acetaminophen, Alfentanil, Allopurinol, Almotriptan, Alprazolam, Amitriptylinem, Amoxapine, Apomorphine, Aripiprazole, Armodafinil, Asenapine, Atomoxetine, Azelastine, Baclofen, Benzbromarone, Benzydamine, Brexpiprazole, Budesonide, Bupivacaine, Buprenorphine, Buprenorphine, Bupropion, Buspirone, Cabergoline, Capsaicin, Carbamazepine, Carbidopa, Carisprodol, Celecoxib, Citalopram, Clobazam, Clonazepam, Clonidine, Clopidogrel, Colchicine, Cyclobenzaprine, Dalteparin, Desvenlafaxine, Dexamfetamine, Dexmethylphenidate, Diazepam, Diclofenac, Disulfiram, Divalproex, Dolasctron, Doxepin, Dronabinol, Droxidopa, Duloxetine, Eletriptan, Entacapone, Escitalopram, Eslicarbazepine, Esomeprazole, Estradiol, Estrogen, Eszopiclone, Ethosuximide, Etodolac, Ezogabine, Febuxostat, Felbamate, Fenbufen, Fentanyl, Flunisolide, Fluorouracil, Fluoxetine, Fluticasone, Fluvoxamine, Formoterol, Fosphenytoin, Frovatriptan, Gabapentin, Granisetron, Guanfacine, Hydrocodone, Hydrocodone, Hydrocortisone, Hydromorphone, Hydroxyzine, Hypericum Extract, Ibuprofen, Indometacin, Ketorolac, Lacosamide, Lamotrigine, Levetiracetam, Levodopa, Levomilnacipran, Levosalbutamol, Lidocaine, Lisdexamfetamine, Lithium, Lorazepam, Lorcaserin, Losartan, Loxapine, Meclizine, Meloxicam, Metaxalone, Methylphenidate, Milnacipran, Mirtazapine, Modafinil, Morphine, Nabilone, Nadolol, Naloxone, Naltrexone, Naproxen, Naratriptan, Nedocromil, Nefazodone, Nitroglycerin, Olanzapine, Ondansetron, Orlistat, Oxaprozin, Oxcarbazepine, Oxybutynin, Oxycodone, Oxymorphone, Palonosetro, Pamidronate, Paroxetine, Perampanel, Phentermine, Phentolamine, Pramipexole, Prasugrel, Prazepam, Prednisone, Pregabalin, Procaine, Promethazine, Propofol, Quetiapine, Ramelteon, Rasagiline, Remifentanil, Risperidone, Rivastigmine, Rizatriptan, Ropinirole, Ropivacaine, Rotigotine, Rufinamide, Salbutamol, Scopolamine, Selegiline, Sertraline, Sodium Oxybate, Strontium, Sufentanil, Sumatriptan, Suvorexant, Tapentadol, Tasimelteon, Temazepam, Testosterone, Tetracaine, Theophylline, Tiagabine, Tiotropium, Tirofiban, Tolcapone, Topiramate, Tramadol, Trazodone, Triazolam, Trimipramine, Valproic acid, Venlafaxine, Vigabatrin, Vilazodone, Vortioxetine, Zaleplon, Zileuton, Ziprasidone, Zolmitriptan, Zolpidem, Norethisterone, Enalapril, Ethinyl Estradiol, Insulin, Memantine, Methamphetamine, Norelgestromine, Pergolide, Ramipril, Tecrine, Timolol, Tolterodine, and Zonisamide. The transdermal membrane can include polypropylene. The transdermal drug delivery device can further include an adhesive for adhering the transdermal drug delivery device to a skin of a patient. A length of the transdermal drug delivery device can be between 60-80 mm, a width can be between 30-45 mm, and a thickness can be between 6-12 mm. A volume of the transdermal drug delivery device can be between 15 and 30 cm³.

In general, in one embodiment, a method of transdermal drug delivery includes: (1) applying a transdermal delivery system to the skin of a patient, the transdermal delivery system including a reservoir, a transdermal membrane, a piston, a control rod with a plurality of teeth, and a rotational cam having first and second camming surfaces; (2) rotating the cam such that the first camming surface moves from a first position that engages with a first tooth of the plurality of teeth to a second position that disengages with the first tooth such that the piston advances and pushes a first dose of a formulation out of the reservoir, onto the transdermal membrane, and to the skin of the patient.

This and other embodiments can include one or more of the following features. The method can further include alternately rotating the cam in a first direction and a second directions so as to deliver sequential doses of the formulation. The method can further include rotating the cam in a same direction so as to deliver sequential doses of formulation. The method can further include engaging a second tooth of the plurality of teeth with the second camming surface so as to stop the formulation from flowing out of the reservoir.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1C show a transdermal delivery device with a rotational plunger.

FIGS. 2A-2D show operation of the rotational plunger of the delivery device of FIGS. 1A-1C.

FIGS. 3A-3E show a transdermal delivery device with a linear plunger.

FIGS. 4A-4D show operation of the linear plunger of the delivery device of FIGS. 3A-3E.

FIGS. 5A-5B show a close-up of the valve system of the delivery device of FIGS. 3A-3E.

FIGS. 6A-6D show a transdermal delivery system with a storage latch.

FIGS. 7A-7B show a transdermal delivery system with an ejection spring.

### DETAILED DESCRIPTION

The present application discloses devices and methods for transdermal delivery of a formulation, e.g., a bioactive agent.

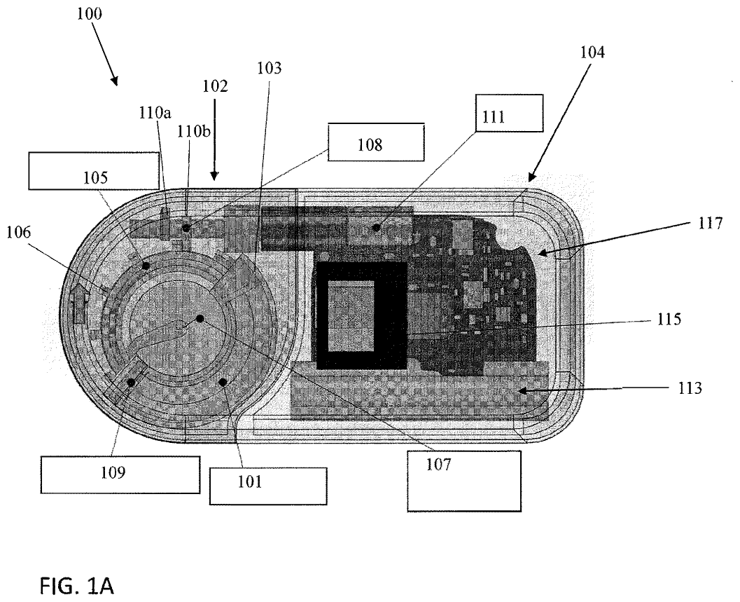

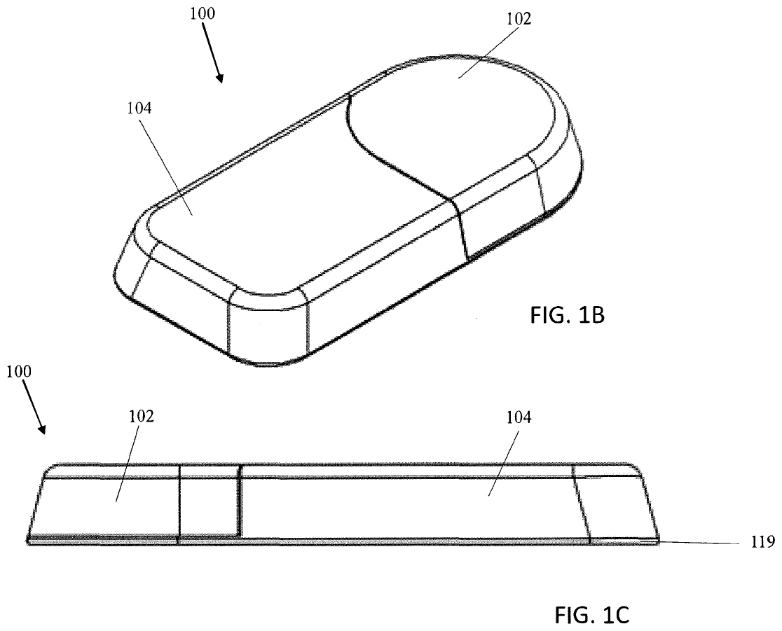

An exemplary transdermal drug delivery device is shown in FIGS. 1A-1C. The delivery device 100 includes a reservoir 101 having a semi-annular (i.e., curved) shape. Further, a rotational plunger including a piston 103 and curved control rod 105 (or drive wheel) can extend at least partially within the reservoir 101. A torsion spring 107 can bias the control rod 105 and piston 103 towards the reservoir 101. The control rod 105 can include a plurality of teeth 106 thereon. Further, a rotatable cam 108 (or cam lock) having two cam surfaces 110a, 110b can be positioned such that the cam surfaces 110a,b can engage with the teeth 106 of the control rod 105. The cam surfaces 110a,b can be semi-circular and can be circumferentially offset relative to one another (e.g., such that there is no circumferential overlap between the two surfaces 110a,b). A valve 109, such as an umbrella valve, can be positioned at the distal end of the reservoir 101 and can prevent fluid from exiting the reservoir 101 until activated by the piston 103. Further, a motor 111 can be connected to the cam 108 so as to rotate the cam 108. The device 100 can further include a printed circuit board (PCB) 117 to control the delivery of fluid as well as a power source, such as a battery 113, and a display 115. A transdermal membrane 119 can be fluidically connected to the reservoir 101 so as to transfer formulation to the skin of the patient during use of the device 100.

The rotational plunger (including the control rod 105 and piston 103) can exert a force on the formulation in the reservoir 101 to expel a dose of the formulation from the reservoir 101. The valve 109, which can be an umbrella or check valve, can be used to prevent leakage of fluid from the reservoir 101 in between doses. The plurality of teeth 106 of the control rod 105 can be contacted by the cam surfaces 110a, 110b of the cam to prevent the piston 103 from moving distally (i.e., further into the reservoir 101) when not activated. The rotational plunger, including the control rod 105 and the piston 103, can be biased distally (i.e., towards the formulation in the reservoir 101) by the compressed torsion spring 107. When the cam surfaces 110a, 110b from the cam 108 no longer restrain the rotating plunger (i.e., when the cam surfaces 110a,b are rotated by the motor 111 so as to release one of the teeth 106 of the control rod 105), the plunger advances distally. Referring to FIG. 1A, as the plunger rotates clockwise (distally into the reservoir 101), a force can be applied to the formulation in the reservoir 101 to expel the solution through the valve 109 and onto the transdermal membrane 119. The next tooth 106 then advances distally and is caught on one of the cam surfaces 110a,b to stop the rotating plunger from moving any further.

The teeth 106 on the control rod 105 can be spaced such that the desired amount of drug is delivered from the reservoir 101. The teeth 106 can be spaced evenly or can have a non-uniform spacing corresponding to the desired drug delivery profile. Further, in some embodiments, a spacing between the teeth 106 can be equivalent to a spacing between the two cam surfaces 110a,b.

In some embodiments, the drug delivery device 100 can include a separable cartridge 102 and control unit 104. The cartridge 102 can, for example, be disposable while the control unit 104 can, for example, be reusable. The cartridge 102 can include the reservoir 101, control rod 105, piston 103, cam 108, spring 107, valve 109, and membrane 119. The control unit 104 can include the motor 111, PCB 117, display 115, and power source 113. In some embodiments, the control unit 104 can further include a user interface.

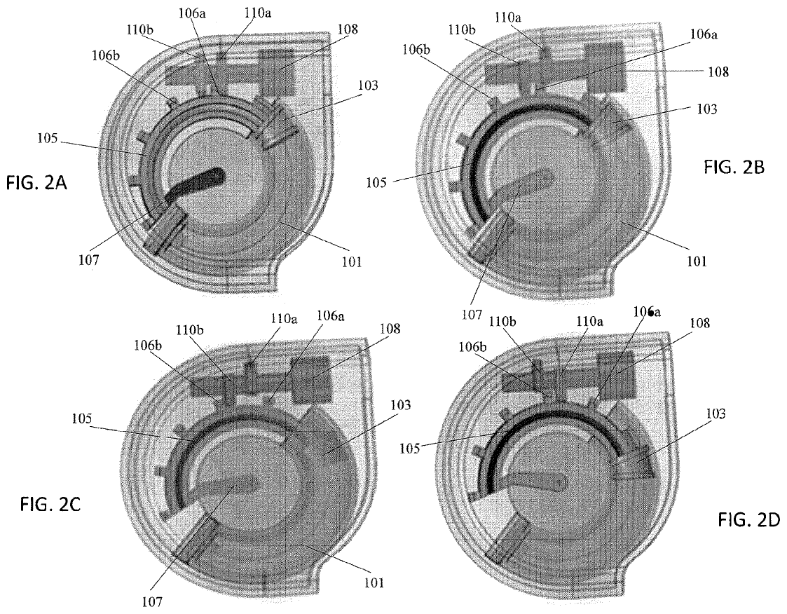

FIGS. 2A-2D illustrate the operation of the rotational plunger (including control rod 105 and plunger 103) and cam 108 to expel fluid from the reservoir 101. As shown in FIG. 2A, the piston 103 can start at a proximal position (e.g., be positioned at the proximal end of the reservoir 101). In this position, the distal cam surface 110a can engage with a first tooth 106a to prevent the torsion spring 107 from moving the control rod 105 and piston 103 distally (i.e. into the reservoir 101). At FIG. 2B, the cam 108 can be rotated such that the distal cam surface 110a disengages from the first tooth 106a. As show in FIG. 2C, the torsion spring 107 can rotate and/or push the control rod 105 and piston 103 into the reservoir 101 to expel a dose of fluid formulation from the reservoir 101. The dose can end (formulation can stop flowing from the reservoir 101) as the second tooth 106b engages with the proximal cam surface 110b. As shown at FIG. 2D, the cam 108 can then be rotated again such that the proximal cam surface 110b disengages from the second tooth 106b. The cam rod 105 and piston 103 can thus rotate and/or move distally to expel fluid formulation from the reservoir 101 until the second tooth 106b engages with the distal cam surface 110a. The process can be continued (e.g., the cam 108 can be further rotated such that the teeth 106 and cam surfaces 110 sequentially engage) to dispel additional doses.

In some embodiments, the cam 108 can be configured to rotate in opposite directions to engage the first and second surfaces 110a,b with the teeth 106 sequentially. In other embodiments, the cam 108 can be configured to rotate in a single direction (e.g., a half rotation) repeatedly to engage the first and second surfaces 110a,b with the teeth 106 sequentially.

The range of motion of the piston 105 for each rotation of the cam 108 can be controlled by varying the spacing between the teeth 106. This feature can be utilized for customizing the dose to be delivered from the device. In one example of non-uniform spacing, the teeth can have a spacing corresponding to delivering bolus volumes of 155 μL, 125 μL, and 80 μL. For a plunger with an internal bore diameter of 4.85 mm, the teeth spacing can be 8.39 mm (155 μL), 6.77 (125 μL) mm, and 4.33 mm (80 μL). The teeth spacing can be adjusted, for example, based on the desired bolus volume and plunger geometry. The drug delivery profile can correspond to a circadian rhythm or a bio-synchronous pattern of a patient using the transdermal drug delivery device. Examples of circadian rhythm or a bio-synchronous drug delivery profile that can be used with the devices described herein are disclosed in US 2015/0283367 and U.S. Pat. No. 8,741,336, the disclosures of each of which are incorporated by reference in its entirety.

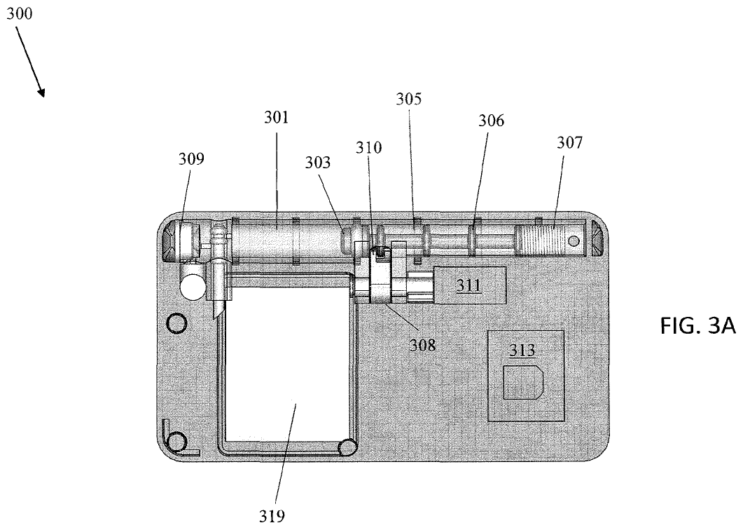

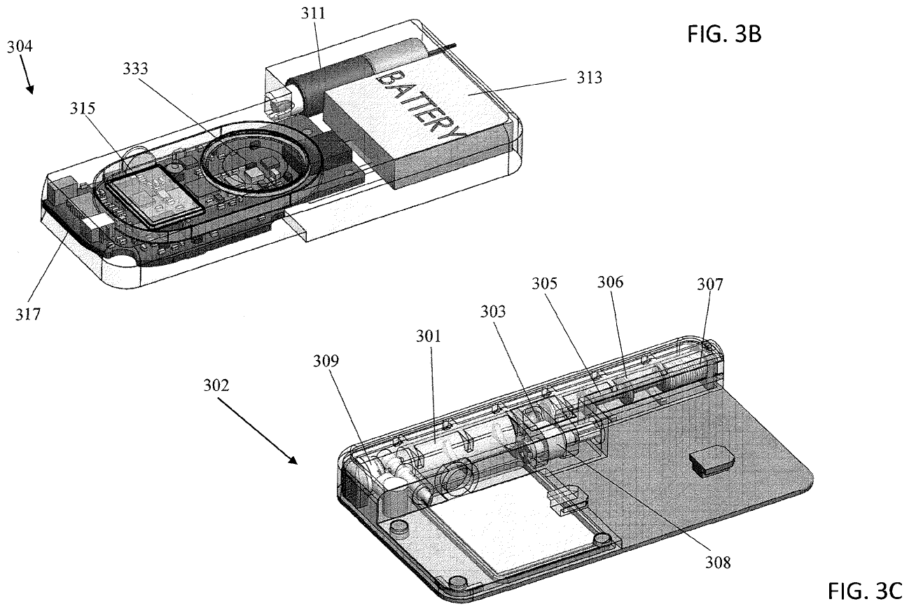

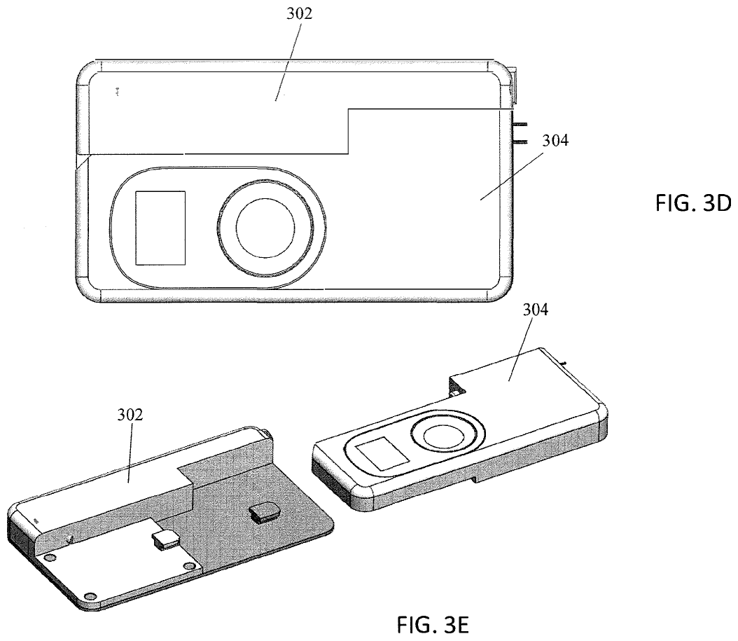

Another exemplary transdermal delivery device is shown in FIGS. 3A-3E. The device 300 is similar to 100 and can include similar features to those described above with respect to device 100. In contrast to claim 100, however, the reservoir 301 and plunger (including rod 305 and piston 303) are substantially linear. The transdermal delivery device 300 thus includes a reservoir 301 having a substantially straight or linear shape. Further, a linear plunger including a piston 303 and straight control rod 305 can extend at least partially within the reservoir 301. A compressed spring 307 can bias the control rod 305 and piston 303 towards the reservoir 301. The control rod 305 can include a plurality of teeth 306 thereon. Further, a rotatable cam 308 (or cam lock) having two cam surfaces 310 can be positioned such that the cam surfaces 310 can engage with the teeth 306 of the control rod 305. The cam surfaces 310 can be semi-circular and can be circumferentially offset relative to one another (e.g., such that there is no circumferential overlap between the two surfaces 310). A valve 309, such as an umbrella valve, can be positioned at the distal end of the reservoir 301 and can prevent fluid from exiting the reservoir 301 until activated by the piston 303. Further, a motor 311 can be connected to the cam 308 so as to rotate the cam 308. The device 300 can further include a printed circuit board (PCB) 317 (see FIG. 3B) to control the delivery of fluid as well as a power source, such as a battery 313, as well as a display 315 and user interface 333 (see FIG. 3B). A transdermal membrane 319 can be fluidically connected to the reservoir 301 so as to transfer fluid to the skin of the patient during use of the device 300.

In some embodiments, the device 300 can include two parts, including a cartridge 302 (shown in FIG. 3C) and a control unit 304 (shown in FIG. 3B), as described with respect to device 100.

The spring 307 can have a compressed configuration such that a force is exerted on the linear plunger (e.g., rod 305 and piston 303). The cam 308, however, can prevent the piston 303 from moving into the reservoir 301 by engaging with the teeth 306 of the control rod 305. When the cam 308 is rotated, a cam surface 310 can disengage with a first tooth 306 on the linear rod 305 to allow the piston 303 to advance until a second tooth 306 on the linear control rod 305 engages with a second cam surface 310 of the cam 308. The advancement of the linear rod 305 and piston 303 pushes on the fluid formulation in the reservoir 301 to expel the formulation from the reservoir 301. The formulation can then travel to the transdermal membrane 319 for release to the skin.

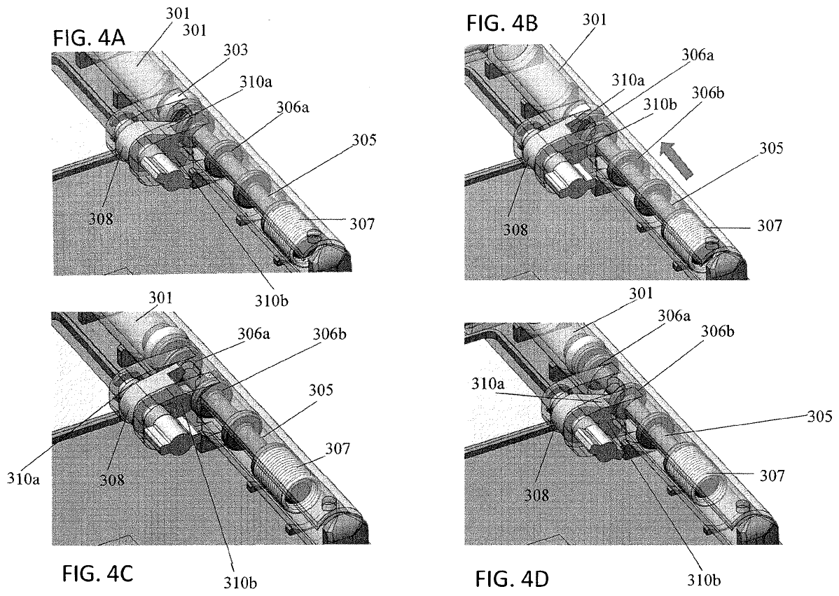

FIGS. 4A-4D illustrate the actuation of the device 300 in accordance with some embodiments. As shown in FIG. 4A, the piston 303 can start at a proximal position (e.g., be positioned at the proximal end of the reservoir 301). In this position, the distal cam surface 310a can engage with a first tooth 306a to prevent the spring 307 from moving the control rod 305 and piston 303 distally (i.e. linearly into the reservoir 301). At FIG. 4B, the cam 308 can be rotated such that the distal cam surface 310a disengages from the first tooth 306a. As show in FIG. 4C, the spring 307 can then push the control rod 305 and piston 303 into the reservoir 301 to expel a dose of fluid formulation from the reservoir 301. The dose can end as the second tooth 306b engages with the proximal cam surface 310b. As shown at FIG. 4D, the cam 308 can be rotated such that the proximal cam surface 310b disengages from the second tooth 306b. The cam rod 305 and piston 303 can thus move distally to expel fluid formulation from the reservoir 301 the second tooth 306b engages with the distal cam surface 310a. The process can be continued (e.g., the cam 308 can be further rotated such that the teeth 306 and cam surfaces 310 sequentially engage) to dispel additional doses.

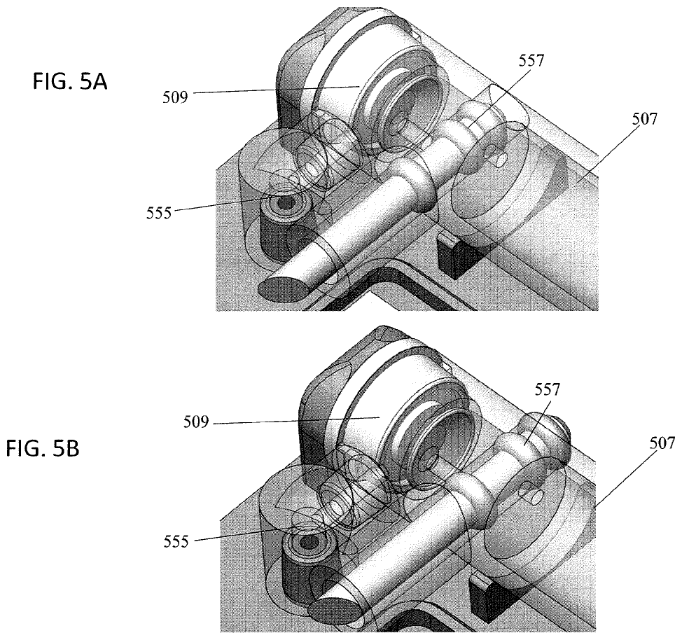

Referring to FIGS. 5A-5B, in some embodiments, a pathway 555 can extend from the reservoir 501 to the transdermal membrane 519. Further, in some embodiments, a spool valve 557 can be used to prevent or minimize evaporative losses from the reservoir 501 during storage. The spool valve 557 can translate to allow the formulation to pass through the spool valve 557, valve 509, and the pathway 555 to the transdermal membrane 519. The spool valve 557 can reduce leakage or evaporative losses from the reservoir during extended storage conditions.

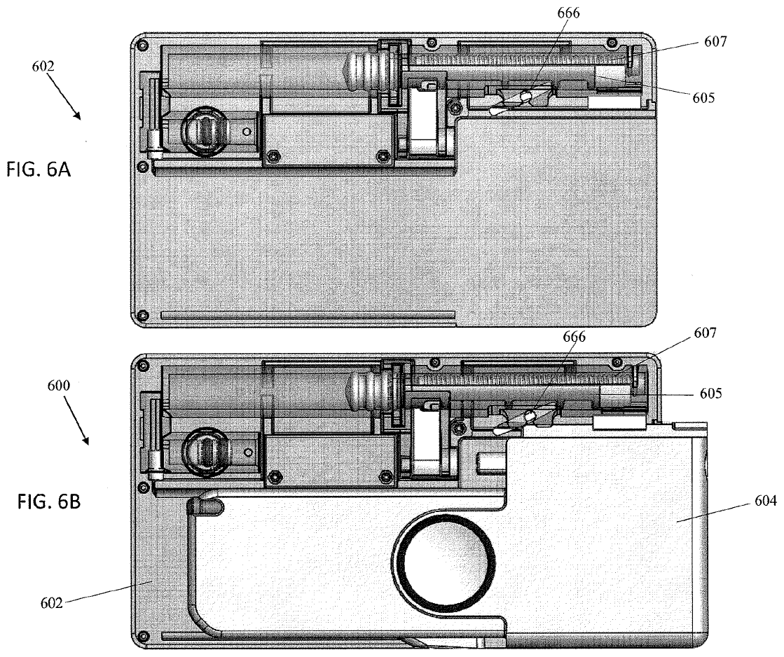

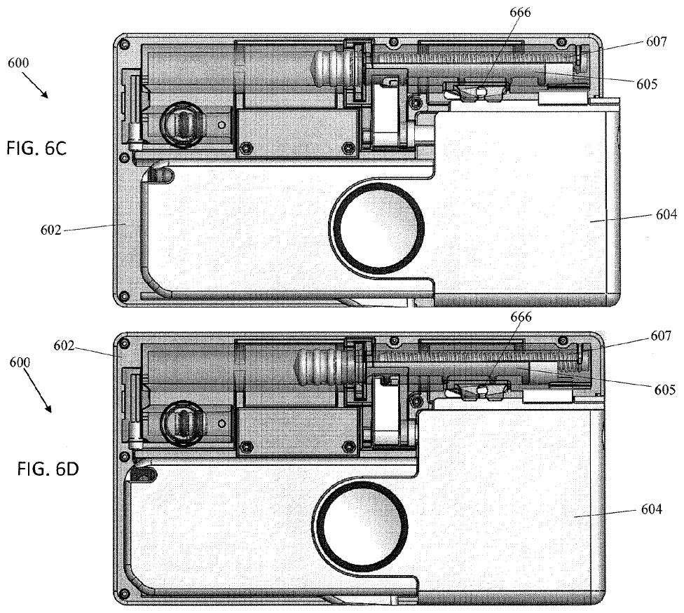

Referring to FIGS. 6A-6D, in some embodiments, a transdermal delivery system 600 similar to device 300 can have a storage latch 666 configured to hold the spring 607 away from the control rod 605 during storage. When the control unit 604 engages with the cartridge 602 (as shown from FIGS. 6B to 6C), the latch 666 can be pushed out of the way of the control rod 605 (by the control unit 604), allowing the control rod 605 to move freely. The latch 666 can thus advantageously help prevent fluid from accidentally be pushed out of the reservoir 601 during storage. Further, in some embodiments, the control unit 604 can push against the distal edge 699 of the control rod 605 (e.g., can move the control rod 2-3 mm) when the control unit 604 is inserted against the cartridge 602 to break the stiction of the control rod 605 and spring 607.

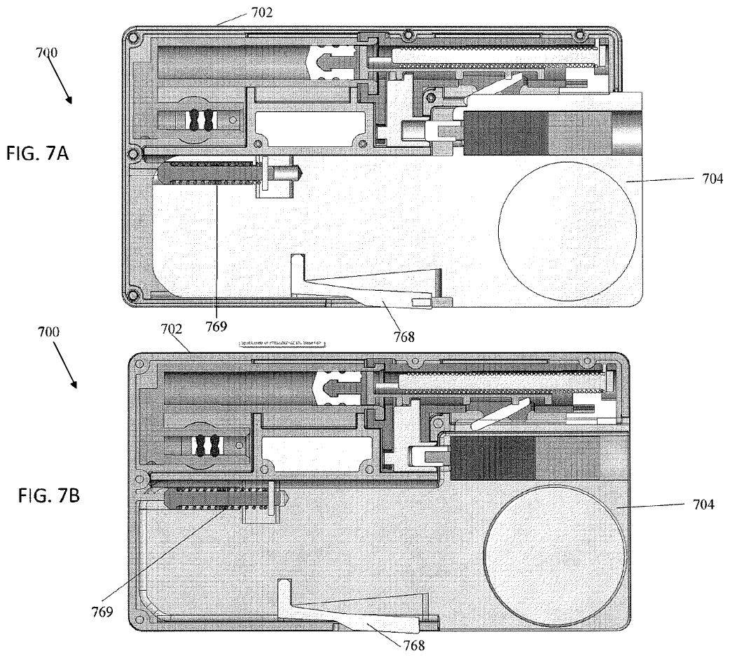

Additionally, in some embodiments, as shown in the device of FIGS. 7A-7B, an ejection spring 769 can be used to help push the control unit 704 and cartridge 702 apart when released (e.g., by release button 768).

The PCBs described herein can include a control unit, processor, wireless data transfer module, and any other electronics used to operate the device. The wireless data transfer module can wirelessly transmit data over a network and/or to and from a computer, such as a hand-held computer (e.g., a smartphone or tablet computer). A software application on the computer can be used to interact with the transdermal drug delivery devices described herein. In some embodiments, sensors can be included on the PCB, such as an accelerometer, temperature sensor, or humidity sensor.

The transdermal drug delivery devices described herein can have various plunger configurations and designs. The size and shape of the reservoir can be configured to work with the plunger configuration. As described above, in some embodiments the plunger and reservoir have a substantially linear configuration while in some embodiments, the plunger and reservoir have a semi-annular or curved configuration.

The transdermal drug delivery devices described herein can have a relatively small profile and volume. The length of the device can be 60-80 mm, such 70 mm, the width of the device can be 30-45 mm, such as 39 mm, and the thickness of the device 100 can be 6-12 mm, such as 9 mm. The volume of the transdermal drug delivery device 100 can be between 15 and 30 cm³, such as about 25 cm³.

The transdermal drug delivery devices described herein can enable the use of a only a small plunger spring. The use of a small spring reduces the complexity, size, and cost of the device. In some embodiments, the plunger spring is adapted to apply a force of about 12 N or less, such as 10N or less, such as 5N or less, such as 3N or less to move the plunger. Where a torsion spring is used, the travel for the torsion spring can be less than about 200° of travel, such as 180° or less.

In some embodiments, a solenoid can be used to activate the cam. In some embodiments, the force used by the motor to actuate the cam is less than about 30 Nm.

In some embodiments, an adhesive can be used with the disposable part for adhering the transdermal drug delivery device to a skin of a patient.

The drug delivery devices described herein can include a transdermal membrane that contacts the wearer's skin. The formulation in the reservoir can be delivered in a controlled amount to the transdermal membrane. The transdermal membrane may be any appropriate material(s) or have any appropriate characteristics that can transfer the bioactive agent across the membrane. The transdermal membrane may be hydrophilic or hydrophobic. The transdermal membrane may have pores having a diameter from 0.010-0.01 μm (e.g., from 0.02 μm-0.05 μm, etc.). The membrane may have porosity over 20%-60% (e.g., from 30%-50%, from 45% to 50%, etc.). In a particular example, the membrane can be made of polypropylene, such as Celgard 2400 polypropylene (e.g., with a thickness around 25 μm such as between 1 μm and 100 μm, with a pore size around 0.043 such as from 0.005 to 0.2 μm, etc. may be used). The material for the transdermal membrane may be chosen, for example, based on the formulation or bioactive agent used or the length of treatment.

A variety of different formulations can be used with the systems described herein. In some embodiments, the formulation includes nicotine. For example, nicotine can be present in the formulation from about 0.5% to about 20% by volume, such as about 0.5% to about 10% by volume, such as about 0.5% to about 5% by volume, such as about 0.5% to about 3% by volume.

Other formulations that can be delivered by the devices described herein include the following drugs and combinations thereof, and modified forms of these drugs including but not limited to salt forms and combinations thereof: Acamprosate, Acetaminophen, Alfentanil, Allopurinol, Almotriptan, Alprazolam, Amitriptylinem, Amoxapine, Apomorphine, Aripiprazole, Armodafinil, Asenapine, Atomoxetine, Azelastine, Baclofen, Benzbromarone, Benzydamine, Brexpiprazole, Budesonide, Bupivacaine, Buprenorphine, Buprenorphine, Bupropion, Buspirone, Cabergoline, Capsaicin, Carbamazepine, Carbidopa, Carisprodol, Celecoxib, Citalopram, Clobazam, Clonazepam, Clonidine, Clopidogrel, Colchicine, Cyclobenzaprine, Dalteparin, Desvenlafaxine, Dexamfetamine, Dexmethylphenidate, Diazepam, Diclofenac, Disulfiram, Divalproex, Dolasetron, Doxepin, Dronabinol, Droxidopa, Duloxetine, Eletriptan, Entacapone, Escitalopram, Eslicarbazepine, Esomeprazole, Estradiol, Estrogen, Eszopiclone, Ethosuximide, Etodolac, Ezogabine, Febuxostat, Felbamate, Fenbufen, Fentanyl, Flunisolide, Fluorouracil, Fluoxetine, Fluticasone, Fluvoxamine, Formoterol, Fosphenytoin, Frovatriptan, Gabapentin, Granisetron, Guanfacine, Hydrocodone, Hydrocodone, Hydrocortisone, Hydromorphone, Hydroxyzine, Hypericum Extract, Ibuprofen, Indometacin, Ketorolac, Lacosamide, Lamotrigine, Levetiracetam, Levodopa, Levomilnacipran, Levosalbutamol, Lidocaine, Lisdexamfetamine, Lithium, Lorazepam, Lorcaserin, Losartan, Loxapine, Meclizine, Meloxicam, Metaxalone, Methylphenidate, Milnacipran, Mirtazapine, Modafinil, Morphine, Nabilone, Nadolol, Naloxone, Naltrexone, Naproxen, Naratriptan, Nedocromil, Nefazodone, Nitroglycerin, Olanzapine, Ondansetron, Orlistat, Oxaprozin, Oxcarbazepine, Oxybutynin, Oxycodone, Oxymorphone, Palonosetro, Pamidronate, Paroxetine, Perampanel, Phentermine, Phentolamine, Pramipexole, Prasugrel, Prazepam, Prednisone, Pregabalin, Procaine, Promethazine, Propofol, Quetiapine, Ramelteon, Rasagiline, Remifentanil, Risperidone, Rivastigmine, Rizatriptan, Ropinirole, Ropivacaine, Rotigotine, Rufinamide, Salbutamol, Scopolamine, Selegiline, Sertraline, Sodium Oxybate, Strontium, Sufentanil, Sumatriptan, Suvorexant, Tapentadol, Tasimelteon, Temazepam, Testosterone, Tetracaine, Theophylline, Tiagabine, Tiotropium, Tirofiban, Tolcapone, Topiramate, Tramadol, Trazodone, Triazolam, Trimipramine, Valproic acid, Venlafaxine, Vigabatrin, Vilazodone, Vortioxetine, Zaleplon, Zileuton, Ziprasidone, Zolmitriptan, Zolpidem, Norethisterone, Enalapril, Ethinyl Estradiol, Insulin, Memantine, Methamphetamine, Norelgestromine, Pergolide, Ramipril, Tecrine, Timolol, Tolterodine, and Zonisamide.

In some embodiments, the formulation used with the delivery devices described herein can include a bioactive agent (e.g., comprising one of the formulations described herein) and a solvent. In such cases, the transdermal membrane can be configured to minimize permeation of the solvent solution while permitting diffusion of a drug or other bioactive agent across the membrane and into contact with the skin. The solvent solution can be removed through a vapor permeable membrane.

In embodiments where the solvent is removed, the removed solvent can be collected in a solvent removal element. An example of a solvent removal element that can be used in the transdermal drug delivery devices described herein is disclosed in U.S. Pat. No. 8,673,346, the disclosure of which is incorporated by reference in its entirety. In some embodiments, the composition of the solvent can be designed and selected to optimize the diffusion of the drug or bioactive agent across the transdermal membrane. In some embodiments, the composition of the solvent can also be chosen in combination with the transdermal membrane to achieve the desired drug or bioactive agent delivery rate. In some embodiments, the solvent recovery element that includes an absorbent to receive and hold the solvent. The solvent recovery element can be part of the disposable part or cartridge. An absorbent for use with a transdermal patch as described herein may be an absorbent gel, blotting paper, paper, other polymer, silica gel or other material that readily soaks up or holds a fluid media such as a solvent liquid or vapor. The absorbent generally behaves as a physical sponge. The absorbent may be any structure or shape, such as a single piece or a plurality of pieces. The absorbent may be an amorphous material or a formed material, and may be a block, a layer, a sheet, a plurality of sheets, a plurality of particles and so on. A desiccant may be used instead or in addition to the absorbent.

The solvent for a bioactive agent may include a single component or multiple components, such as alcohol, water, or another solvent that readily vaporizes. One or more than one component may vaporize and be absorbed by absorbent. In some embodiments, the solvent solution includes water, alcohol, and a drug or bioactive agent. In some embodiments, the alcohol can be one or more of isopropanol, ethanol, and methanol. The solvent solution can also include one or more of a: surfactant, excipient, or other component intended to enhance permeation or decrease skin sensitivity or skin reaction. The solvent solution can have a ratio of water to alcohol of about 40:60 to about 60:40. The solvent solution can have a ratio of water to alcohol of about 45:55 to about 55:45. The solvent solution can have a ratio of water to alcohol of about 46:54 to about 54:46. The solvent solution can have a ratio of water to alcohol of about 47:53 to about 53:47. The solvent solution can have a ratio of water to alcohol of about 48:52 to about 52:48. The solvent solution can have a ratio of water to alcohol of about 49:51 to about 51:49.

In some embodiments, the formulation (e.g., nicotine or any of the other formulations described herein) used with the devices described herein can be provided for smoking cessation or to treat Parkinson's and other conditions.

The systems described herein can efficiently deliver substantially all of the formulation (e.g., at least 90%, at least 95%, at least 97%, at least 98%, or at least 99% of the formulation) across the transdermal membrane into contact with the wearer's skin.

The systems described herein can be configured to provide a single bolus or to provide a plurality of boluses (such as 2 or more, 3 or more, 4 or more, or 5 or more boluses).

Any feature or element described herein with respect to one embodiment can be combined with, or substituted for, any feature or element described with respect to another embodiment. Further, transdermal drug delivery systems are described in US 2016/0220798 titled “Drug Delivery Methods and Systems,” the entirety of which is incorporated by reference herein in its entirety. Any feature or element described with respect to an embodiment herein can be combined with, or substituted for, any feature or element described in US 2016/0220798.

When a feature or element is herein referred to as being “on” another feature or clement, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A transdermal drug delivery device comprising:
a reservoir configured to hold a formulation therein, the reservoir comprising a curved shape;
a transdermal membrane configured to allow the formulation from the reservoir to pass therethrough;
a piston configured to move into the reservoir;
a curved control rod attached to the piston, the curved control rod including a plurality of teeth thereon;
a spring configured to apply rotational force to the control rod toward the reservoir; and
a rotational cam having a first camming surface and a second camming surface, the first camming surface and the second camming surface configured to engage with the plurality of teeth,
wherein the rotational cam is rotatable a first distance in a first direction to cause the first camming surface to disengage with a first tooth of the plurality of teeth and the second camming surface to engage with a second tooth of the plurality of teeth, and wherein the rotational cam is rotatable a second distance in the first direction to cause the second camming surface to disengage from the second tooth of the plurality of teeth.

2. The transdermal drug delivery device of claim 1, wherein the curved control rod is configured to allow the spring to advance the piston into the reservoir and expel the formulation onto the transdermal membrane when the first camming surface disengages with the first tooth and the second camming surface engages with the second tooth.

3. The transdermal drug delivery device of claim 2, wherein the curved control rod is further configured to allow the spring to further advance the piston into the reservoir and expel more formulation onto the transdermal membrane when the second camming surface disengages with the second tooth.

4. The transdermal drug delivery device of claim 1, wherein the reservoir comprises a semi-annular shape.

5. The transdermal drug delivery device of claim 1, wherein the piston comprises a curved shape.

6. The transdermal drug delivery device of claim 1, further comprising a motor configured to rotate the rotational cam.

7. The transdermal drug delivery device of claim 6, wherein the motor is further configured to rotate a first direction to rotate the rotational cam the first distance and to rotate a second direction to rotate the rotational cam the second distance.

8. The transdermal drug delivery device of claim 1, further comprising a storage latch configured to hold the spring away from the control rod during storage.

9. The transdermal drug delivery device of claim 1, wherein a spacing between the plurality of teeth on the control rod defines a teeth spacing pattern, and wherein the teeth spacing pattern corresponds to a drug delivery profile of the transdermal drug delivery device.

10. The transdermal drug delivery device of claim 1, wherein a drug delivery profile corresponds to a circadian rhythm or a bio-synchronous pattern of a patient using the transdermal drug delivery device.

11. The transdermal drug delivery device of claim 1, wherein the rotational cam is further configured to rotate in a first direction to cause the first camming surface to disengage with the first tooth of the plurality of teeth and the second camming surface to engage with the second tooth, and wherein the rotational cam is further configured to rotate in a second direction to cause the second camming surface to disengage from the second tooth of the plurality of teeth and cause the piston to move further into the reservoir to expel more of the formulation.

12. The transdermal drug delivery device of claim 11, wherein the rotational cam is configured to rotate alternately in the first and second directions to allow sequential bolus deliveries of the formulation.

13. The transdermal drug delivery device of claim 1, further comprising a valve along a pathway between the reservoir and the transdermal membrane.

14. The transdermal drug delivery device of claim 1, wherein the formulation is selected from the group consisting of: nicotine, Acamprosate, Acetaminophen, Alfentanil, Allopurinol, Almotriptan, Alprazolam, Amitriptylinem, Amoxapine, Apomorphine, Aripiprazole, Armodafinil, Asenapine, Atomoxetine, Azelastine, Baclofen, Benzbromarone, Benzydamine, Brexpiprazole, Budesonide, Bupivacaine, Buprenorphine, Buprenorphine, Bupropion, Buspirone, Cabergoline, Capsaicin, Carbamazepine, Carbidopa, Carisprodol, Celecoxib, Citalopram, Clobazam, Clonazepam, Clonidine, Clopidogrel, Colchicine, Cyclobenzaprine, Dalteparin, Desvenlafaxine, Dexamfetamine, Dexmethylphenidate, Diazepam, Diclofenac, Disulfiram, Divalproex, Dolasetron, Doxepin, Dronabinol, Droxidopa, Duloxetine, Eletriptan, Entacapone, Escitalopram, Eslicarbazepine, Esomeprazole, Estradiol, Estrogen, Eszopiclone, Ethosuximide, Etodolac, Ezogabine, Febuxostat, Felbamate, Fenbufen, Fentanyl, Flunisolide, Fluorouracil, Fluoxetine, Fluticasone, Fluvoxamine, Formoterol, Fosphenytoin, Frovatriptan, Gabapentin, Granisetron, Guanfacine, Hydrocodone, Hydrocodone, Hydrocortisone, Hydromorphone, Hydroxyzine, Hypericum Extract, Ibuprofen, Indometacin, Ketorolac, Lacosamide, Lamotrigine, Levetiracetam, Levodopa, Levomilnacipran, Levosalbutamol, Lidocaine, Lisdexamfetamine, Lithium, Lorazepam, Lorcaserin, Losartan, Loxapine, Meclizine, Meloxicam, Metaxalone, Methylphenidate, Milnacipran, Mirtazapine, Modafinil, Morphine, Nabilone, Nadolol, Naloxone, Naltrexone, Naproxen, Naratriptan, Nedocromil, Nefazodone, Nitroglycerin, Olanzapine, Ondansetron, Orlistat, Oxaprozin, Oxcarbazepine, Oxybutynin, Oxycodone, Oxymorphone, Palonosetro, Pamidronate, Paroxetine, Perampanel, Phentermine, Phentolamine, Pramipexole, Prasugrel, Prazepam, Prednisone, Pregabalin, Procaine, Promethazine, Propofol, Quetiapine, Ramelteon, Rasagiline, Remifentanil, Risperidone, Rivastigmine, Rizatriptan, Ropinirole, Ropivacaine, Rotigotine, Rufinamide, Salbutamol, Scopolamine, Selegiline, Sertraline, Sodium Oxybate, Strontium, Sufentanil, Sumatriptan, Suvorexant, Tapentadol, Tasimelteon, Temazepam, Testosterone, Tetracaine, Theophylline, Tiagabine, Tiotropium, Tirofiban, Tolcapone, Topiramate, Tramadol, Trazodone, Triazolam, Trimipramine, Valproic acid, Venlafaxine, Vigabatrin, Vilazodone, Vortioxetine, Zaleplon, Zileuton, Ziprasidone, Zolmitriptan, Zolpidem, Norethisterone, Enalapril, Ethinyl Estradiol, Insulin, Memantine, Methamphetamine, Norelgestromine, Pergolide, Ramipril, Tecrine, Timolol, Tolterodine, and Zonisamide.

15. The transdermal drug delivery device of claim 1, further comprising a control unit configured to control rotation of the rotational cam.

16. The transdermal drug delivery device of claim 1, further comprising a disposable part and a reusable part.

17. The transdermal drug delivery device of claim 1, wherein the control rod is configured to deliver non-uniform bolus volumes.

18. The transdermal drug delivery device of claim 1, wherein the plurality of teeth are configured to deliver two or more bolus volumes.

19. A method of transdermal drug delivery, the method comprising:
applying a transdermal delivery system to skin of a patient, the transdermal delivery system including a reservoir comprising a curved shape, a transdermal membrane, a piston, a curved control rod having a plurality of teeth, a spring to apply a rotational force to the control rod, and a rotational cam having first and second camming surfaces configured to engage the control rod; and
rotating the rotational cam a first distance in a first direction to cause the first camming surface to disengage with a first tooth of the plurality of teeth and the second camming surface to engage with a second tooth of the plurality of teeth, and wherein the rotational cam is rotatable a second distance in the first direction to cause the second camming surface to disengage from the second tooth of the plurality of teeth.

20. The method of claim 19. further comprising alternately rotating the cam in a first direction and a second direction so as to deliver sequential doses of a formulation.
